(19) United States
(12) Patent Application Publication
(10) Pub. No.: US 2026/0252598 A1
(43) Pub. Date: Aug. 27, 2026

(54) QUESTION ANSWERING DEVICE, QUESTION ANSWERING METHOD, AND RECORDING MEDIUM

(71) Applicant: NEC Corporation, Tokyo (JP)

(72) Inventor: Kazeto YAMAMOTO, Tokyo (JP)

(73) Assignee: NEC Corporation, Tokyo (JP)

(21) Appl. No.: 19/161,412

(22) PCT Filed: Mar. 13, 2023
(86) PCT No.: PCT/JP2023/009520

§ 371 Date: Sep. 2, 2025

(51) Int. Cl. G06F 16/3329; G06F 16/334; G06T 11/26; G16H 20/00

(52) U.S. Cl. CPC G06F 16/33295; G06F 16/3344; G06T 11/26; G16H 20/00

(57) ABSTRACT

In a question answering device, a query acquisition acquires a query. A starting point phrase determination determines a starting point phrase related to the query from a plurality of phrases included in causal relationship data. An inference type determination means determines an inference type based on the query. A search means searches for a phrase by tracing a causal relationship included in the causal relationship data from the starting point phrase in a search procedure according to the determined inference type. An output means outputs a phrase obtained by the search as an answer to the query.

### TECHNICAL FIELD

The present disclosure relates to a question answering technique.

### BACKGROUND ART

A question answering system that accepts a question in a natural language from a user and returns an answer to the question is known. Patent Document 1 discloses a question answering device that outputs an answer to a question sentence in consideration of causal relationships between the question sentence and answer candidates.

### PRECEDING TECHNICAL REFERENCES

### Patent Document

Patent Document 1: Japanese Laid-open Patent Publication No. 2008-282366

### SUMMARY

### Problem to be Solved by the Invention

In the case of generating an answer to a question using causal relationship knowledge, it is required to efficiently generate and output the answer to the question based on an enormous amount of causal relationship knowledge.

One object of the present disclosure is to provide a question answering device capable of efficiently generating and outputting an answer to a question based on causal relationship knowledge.

### Means for Solving the Problem

According to an example aspect of the present disclosure, there is provided a question answering device including:
a query acquisition means configured to acquire a query;
a starting point phrase determination means configured to determine a starting point phrase related to the query from a plurality of phrases included in causal relationship data;
an inference type determination means configured to determine an inference type based on the query;
a search means configured to search for a phrase by tracing a causal relationship included in the causal relationship data from the starting point phrase in a search procedure according to the determined inference type; and
an output means configured to output a phrase obtained by the search as an answer to the query.

According to another example aspect of the present disclosure, there is provided a question answering method executed by a computer, the question answering method including:
acquiring a query;
determining a starting point phrase related to the query from a plurality of phrases included in causal relationship data;
determining an inference type based on the query;
searching for a phrase by tracing a causal relationship included in the causal relationship data from the starting point phrase in a search procedure according to the determined inference type; and
outputting a phrase obtained by the search as an answer to the query.

According to a further example aspect of the present disclosure, there is provided a recording medium storing a program, the program causing a computer to perform a process including: acquiring a query;
determining a starting point phrase related to the query from a plurality of phrases included in causal relationship data;
determining an inference type based on the query;
searching for a phrase by tracing a causal relationship included in the causal relationship data from the starting point phrase in a search procedure according to the determined inference type; and
outputting a phrase obtained by the search as an answer to the query.

### BRIEF DESCRIPTION OF THE DRAWINGS

FIG. 1 illustrates an overall configuration of a question answering system according to a first example embodiment.

FIG. 2 is a block diagram illustrating a hardware configuration of a question answering device.

FIG. 3 is a block diagram illustrating a functional configuration of the question answering device.

FIG. 4 schematically illustrates an example of causal relationship knowledge stored in a causal relationship database.

FIG. 5 illustrates an example of causal inference.

FIG. 6 illustrates an example of inverse causal inference.

FIG. 7 illustrates an example of problem-solution inference.

FIG. 8 illustrates an example of solution-problem inference.

FIG. 9 is a flowchart of inference processing by the question answering device according to the first example embodiment.

FIG. 10 is a block diagram illustrating a functional configuration of a question answering device according to a second example embodiment.

FIG. 11 is a flowchart of inference processing by the question answering device according to the second example embodiment.

FIG. 12 illustrates a display example of an answer graph output by the question answering device according to the second example embodiment.

FIG. 13 illustrates another display example of the answer graph output by the question answering device according to the second example embodiment.

FIG. 14 is a block diagram illustrating a configuration of a medical knowledge generation system according to a third example embodiment.

FIG. 15 is a block diagram illustrating a configuration of a question answering system according to the third example embodiment.

FIG. 16 is a block diagram illustrating a functional configuration of a question answering device according to a fourth example embodiment.

FIG. 17 is a flowchart of processing by the question answering device according to the fourth example embodiment.

### EXAMPLE EMBODIMENTS

Hereinafter, preferred example embodiments of the present disclosure will be described with reference to the drawings.

### First Example Embodiment

### Overall Configuration

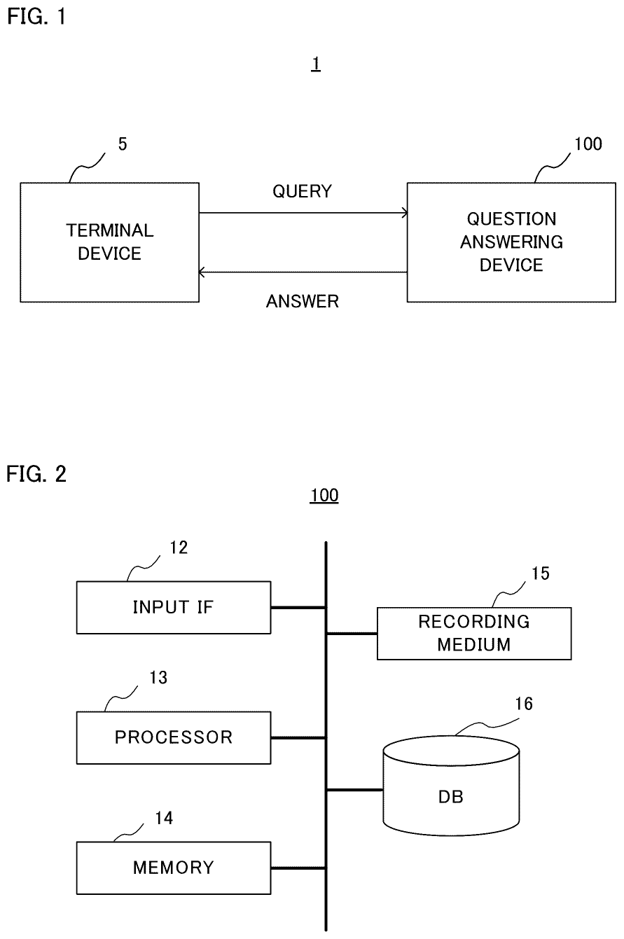

FIG. 1 illustrates an overall configuration of a question answering system according to a first example embodiment. A question answering system 1 includes a terminal device 5 of a user and a question answering device 100. The user operates the terminal device 5 to transmit a question (hereinafter, a question is referred to as a “query”) to the question answering device 100. The query is described in a natural language. The question answering device 100 receives the query from the user. The question answering device 100 generates an answer to the query using causal relationship knowledge defining a relationship between a plurality of phrases having a causal relationship, and transmits the answer to the terminal device 5 of the user.

### Basic Principle

Next, a basic principle of the question answering device 100 will be described. The question answering device 100 searches for an answer to a query from a user based on causal relationship knowledge prepared in advance. The causal relationship knowledge defines a causal relationship between a plurality of sentences (hereinafter, sentences are also referred to as “phrases”) described in a natural language. The question answering device 100 first determines a phrase related to the query of the user among the plurality of phrases included in the causal relationship knowledge as a starting point phrase. The question answering device 100 then searches for a phrase having a causal relationship with the query of the user by tracing a causal relationship included in the causal relationship knowledge from the starting point phrase, and generates an answer to the query. Note that the question answering device 100 may search for a phrase by tracing a specific semantic relationship to be described later in addition to the causal relationship. As a result, the question answering device 100 can generate an answer having a causal relationship with the query of the user based on the causal relationship knowledge prepared in advance. Note that a process of deriving an answer to the query of the user based on the causal relationship knowledge is referred to as “inference”.

### Hardware Configuration

FIG. 2 is a block diagram illustrating a hardware configuration of the question answering device 100. As illustrated, the question answering device 100 includes an interface (IF) 12, a processor 13, a memory 14, a recording medium 15, and a database (DB) 16.

The IF 12 acquires a query input by a user by communicating with an external device. The IF 12 transmits an answer generated by the question answering device 100 to the external device. Specifically, in a case where the user transmits the query to the question answering device 100 using the terminal device 5, the question answering device 100 receives the query through the IF 12, and transmits an answer related to the query to the terminal device 5 of the user through the IF 12.

The processor 13 is a computer such as a central processing unit (CPU), and controls the entire question answering device 100 by executing a program prepared in advance. Specifically, it is possible to use, as the processor 13, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, a combination of these, or the like. Specifically, the processor 13 executes inference processing of generating an answer to the query.

The memory 14 includes a read only memory (ROM), a random access memory (RAM), and the like. The memory 14 stores various programs executed by the processor 13. The memory 14 is also used as a working memory during execution of various types of processing by the processor 13.

The recording medium 15 is a non-volatile and non-transitory recording medium such as a disk-shaped recording medium or a semiconductor memory, and is attachable to and detachable from the question answering device 100. The recording medium 15 records various programs executed by the processor 13. When the question answering device 100 executes various types of processing, a program recorded in the recording medium 15 is loaded into the memory 14, and is executed by the processor 13.

The DB 16 stores causal relationship knowledge used in generating an answer to the query. The DB 16 may store a machine learning model or the like used in inference processing to be described later.

In addition to the above, the question answering device 100 may include a display device such as a liquid crystal display or a projector, and an input device such as a keyboard or a mouse. The display device and input device are used, for example, by an administrator of the question answering device 100 to perform necessary management.

### Functional Configuration

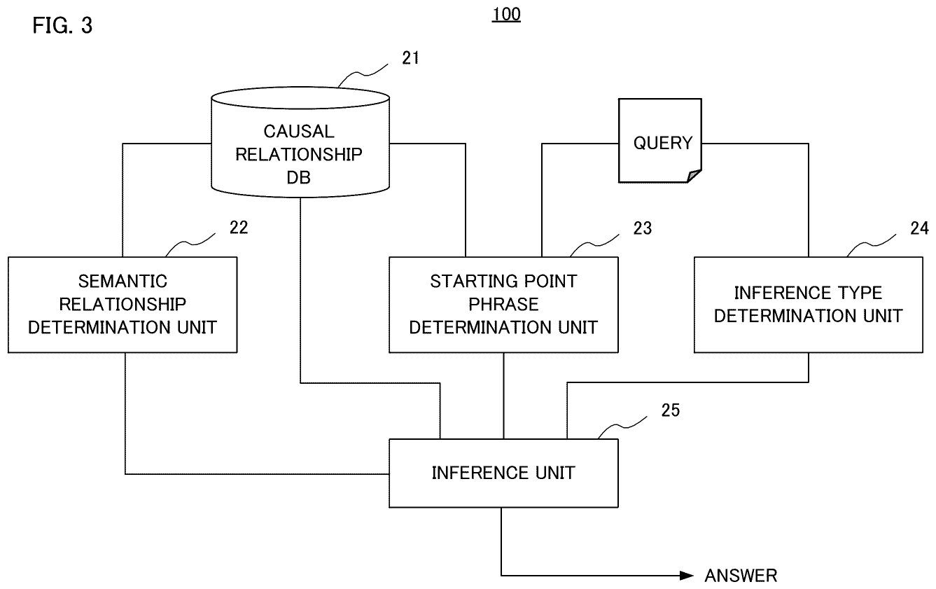

FIG. 3 is a block diagram illustrating a functional configuration of the question answering device 100. The question answering device 100 functionally includes a causal relationship DB 21, a semantic relationship determination unit 22, a starting point phrase determination unit 23, an inference type determination unit 24, and an inference unit 25.

The causal relationship DB 21 stores causal relationship knowledge between a plurality of phrases. The causal relationship knowledge is an example of causal relationship data, and is knowledge data extracted from a large number of documents, Web documents, and the like. In the present example embodiment, the causal relationship knowledge is defined as including the following elements having a causal relationship.
“A natural language sentence expressing a cause (cause phrase)”
“A natural language sentence expressing a result (result phrase)”

The causal relationship knowledge is prepared in advance as a plurality of pairs of a cause phrase and a result phrase (hereinafter, such a pair is also referred to as a “causal relationship pair”) and stored in the causal relationship DB 21.

In the present example embodiment, a “causal relationship” refers to a correlation such as “if one happens, the other does too”. Therefore, in the present example embodiment, causal relationships include the following relationships.

### (1) Causal Relationship in Narrow Sense

A causal relationship in a narrow sense refers to a relationship in which one causes the other. For example, “The number of births is reduced.”→“The aging of the population is progressing.”.

### (2) Anteroposterior Relationship

An anteroposterior relationship refers to a temporal relationship in which one occurs and then the other occurs. For example, “going to a restaurant”→“ordering a meal”.

### (3) Entailment Relationship

An entailment relationship refers to a relationship in which one statement logically implies another. For example, “The state of a field is managed by AI.”→“The agriculture is digitized.”.

As described above, the causal relationship DB 21 stores a plurality of causal relationship pairs, but may further store the following additional information in the causal relationship DB 21.

### (i) Source Information of Causal Relationship

Source information of a causal relationship is, for example, information indicating an information source such as a document or a Web document in which a certain causal relationship pair is described. When an answer to a query is presented to a user, source information of a causal relationship can be used as information indicating a basis on which the answer is selected.

### (ii) Sub-Classification of Causal Relationship

A subcategory of a causal relationship is a classification indicating a more detailed relationship between phrases constituting a causal relationship pair. Examples of subcategories of causal relationships include a “relationship between a problem and a solution to the problem (problem-solution relationship)”, a “chronological order relationship”, and the like. The relationships included in the “causal relationships” in the present example embodiment as described above (the causal relationship in a narrow sense, the anteroposterior relationship, and the entailment relationship) may be used as subcategories.

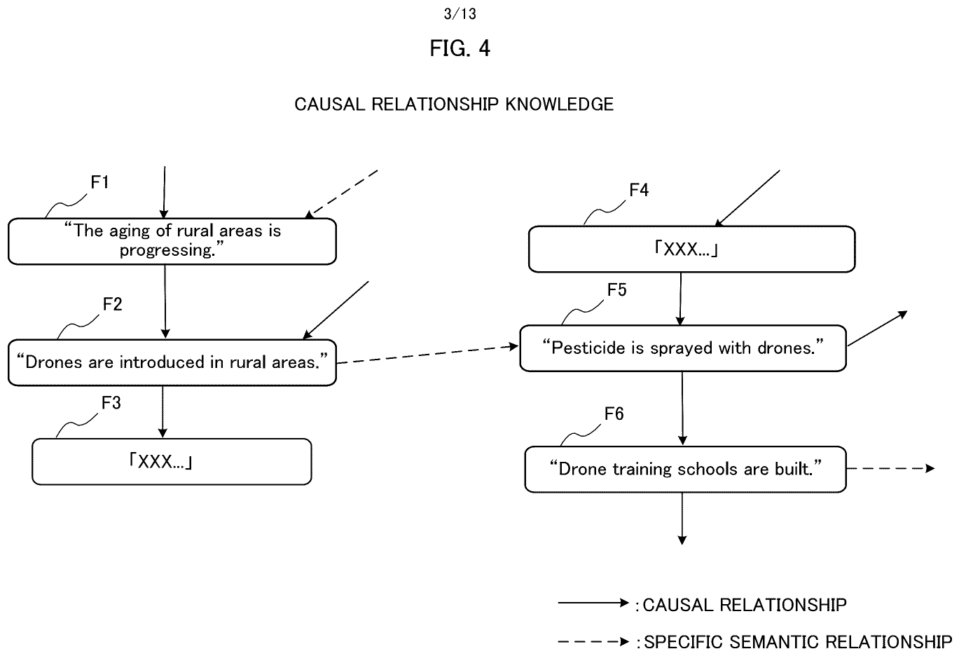

FIG. 4 schematically illustrates an example of the causal relationship knowledge stored in the causal relationship DB 21. FIG. 4 illustrates the causal relationship knowledge in a graph structure for convenience of description. Each of phrases F1 to F6 corresponds to a node of the graph structure, and a causal relationship between phrases is indicated by a solid arrow (edge). For example, the phrases F1 and F2 constitute a causal relationship pair of “The aging of rural areas is progressing.”→“Drones are introduced in rural areas.”. Note that FIG. 4 is obtained by extracting a part of the causal relationship knowledge including a large number of causal relationship pairs.

The data structure of the causal relationship knowledge stored in the causal relationship DB 21 is not limited to the graph structure as described above. For example, the causal relationship DB 21 may store the causal relationship knowledge as a list of a large number of causal relationship pairs.

The semantic relationship determination unit 22 extracts a pair of phrases related to a specific semantic relationship (hereinafter, such a pair is also referred to as a “specific semantic relationship pair”) from among the plurality of phrases included in the causal relationship knowledge stored in the causal relationship DB 21. Here, the “specific semantic relationship” is a relationship in which meanings of phrases are close to each other, and specifically includes an entailment relationship, a synonym relationship, a similar meaning relationship, and the like. The entailment relationship is a relationship in which one of two phrases entails the other. The synonym relationship is a relationship in which the meanings of two phrases are the same, and the similar meaning relationship is a relationship in which the meanings of two phrases are similar.

The reason for extracting a specific semantic relationship pair from the plurality of phrases included in the causal relationship knowledge in this way is as follows. As described above, the causal relationship pairs constituting the causal relationship knowledge are phrase pairs described in a natural language, and thus the causal relationship knowledge includes a plurality of phrases having an entailment relationship and a plurality of phrases describing substantially the same or similar contents. Therefore, flexibly connecting such phrases having close meanings and expanding causal relationship knowledge makes it possible to infer an answer from a query using wider causal relationship knowledge.

In FIG. 4, a dashed arrow indicates an edge connecting a specific semantic relationship pair. In the example of FIG. 4, the phrase F2 “Drones are introduced in rural areas.” and the phrase F5 “Pesticide is sprayed with drones.” have an entailment relationship and correspond to a specific semantic relationship pair. Thus, although there is no causal relationship between the phrases F2 and F5 in the original causal relationship knowledge, the phrases F2 and F5 are defined as a specific semantic relationship pair. At the time of inference based on a query, the question answering device 100 searches for an answer to the query by tracing a causal relationship and a specific semantic relationship. In this way, in the example of FIG. 4, the question answering device 100 can make a search by tracing more phrases such as the phrases F1→F2→F5→F6. This makes it possible to infer the answer to the query using wider causal relationship knowledge.

As an example, the semantic relationship determination unit 22 can determine whether two phrases have a specific semantic relationship by using feature vectors of the phrases to be determined. For example, the semantic relationship determination unit 22 converts each of the two phrases into a feature vector, and calculates the similarity or the distance between the feature vectors. In a case where the similarity or the distance between the two feature vectors is equal to or less than a predetermined threshold value, the semantic relationship determination unit 22 determines that these phrases have a specific semantic relationship. The semantic relationship determination unit 22 outputs the extracted specific semantic relationship pair to the inference unit 25.

The starting point phrase determination unit 23 determines a starting point phrase related to the query input by the user with reference to the causal relationship knowledge stored in the causal relationship DB 21. The “starting point phrase” is a phrase that is a starting point at the time of tracing the causal relationship knowledge based on the query input by the user in the inference of the answer to the query. Specifically, the starting point phrase determination unit 23 analyzes the meaning of the query of the user. The starting point phrase determination unit 23 then determines, as the starting point phrase, a phrase having a meaning close to that of the query of the user among the plurality of phrases included in the causal relationship knowledge. For example, in a case where the query of the user is “What will happen if the aging of rural areas progresses?”, the starting point phrase determination unit 23 searches the causal relationship knowledge for a phrase having substantially the same meaning as the query or a phrase having a meaning closer to that of the query than a predetermined standard. For example, in a case where there is a phrase “aging of rural areas” in the causal relationship knowledge stored in the causal relationship DB 21, the starting point phrase determination unit 23 determines the phrase as the starting point phrase. In one example, similarly to the semantic relationship determination unit 22 described above, the starting point phrase determination unit 23 may determine, as the starting point phrase, a phrase in which the similarity between the feature vectors of the phrases is higher than a predetermined reference value or a phrase in which the distance between the feature vectors is closer than a predetermined reference value. The starting point phrase determination unit 23 outputs the determined starting point phrase to the inference unit 25.

Note that the number of starting point phrases determined by the starting point phrase determination unit 23 is not limited to one for one query, and may be plural. For example, in a case where the query of the user is “problems of rural areas”, the starting point phrase determination unit 23 may set, as starting point phrases, a plurality of phrases close to the problems of rural areas among the plurality of phrases included in the causal relationship knowledge.

The inference type determination unit 24 determines an inference type based on the query input by the user. Here, the “inference type” indicates a procedure of searching for a phrase by tracing the causal relationship knowledge at the time of inferring the answer related to the query based on the causal relationship knowledge. Specific examples of inference types include the following types.

### (A) Causal Inference

The causal inference is an inference type for a query such as “What will happen if XX?”, and is a search method of tracing a causal relationship and a specific semantic relationship in a forward direction in the causal relationship knowledge. Note that the forward direction is, for example, the direction of “cause”→“result”.

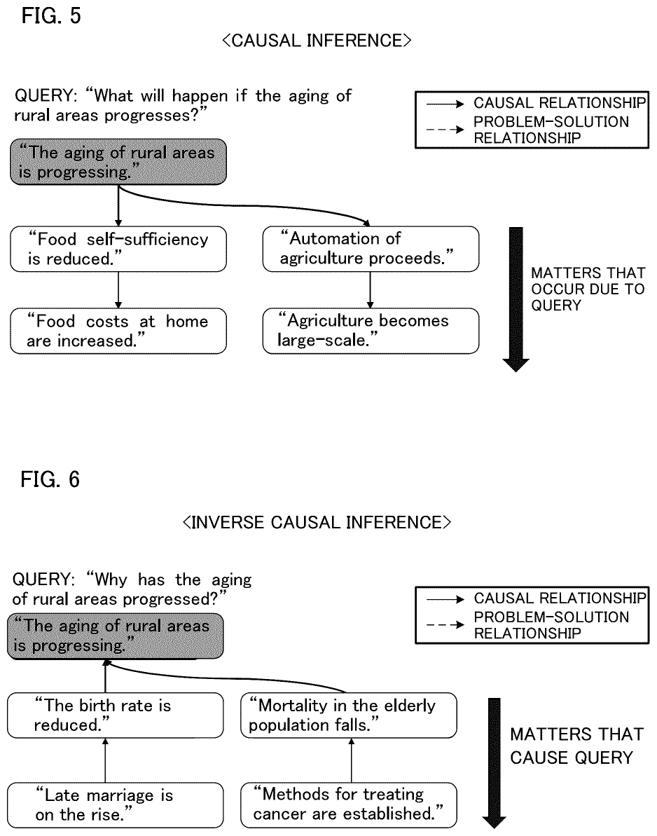

FIG. 5 illustrates an example of the causal inference. Note that a solid arrow in the drawing indicates a causal relationship, and a broken arrow indicates a problem-solution relationship. The causal inference infers matters that occur due to a query. In the example of FIG. 5, a phrase “Food self-sufficiency is reduced.” is obtained based on a query “The aging of rural areas is progressing.”. Furthermore, a phrase “Food costs at home are increased.” is obtained based on the phrase “Food self-sufficiency is reduced.”.

### (B) Inverse Causal Inference

The inverse causal inference is an inference type for a query such as “What is the cause of XX?”, and is a search method of tracing a causal relationship and a specific semantic relationship in a backward direction. The backward direction is, for example, a direction of “result”→“cause”.

FIG. 6 illustrates an example of the inverse causal inference. Note that a solid arrow in the drawing indicates a causal relationship, and a broken arrow indicates a problem-solution relationship. The inverse causal inference infers matters that cause a query. In the example of FIG. 6, a phrase “The birth rate is reduced.” is obtained based on a query “The aging of rural areas is progressing.”. Furthermore, a phrase “Late marriage is on the rise.” is obtained based on the phrase “The birth rate is reduced.”.

### (C) Combination Inference

The combination inference is a method combining the causal inference and the inverse causal inference described above, and includes, for example, the following

### (C1) Problem-Solution Inference

The problem-solution inference is an inference type for a query such as “What should be done to solve XX?”, and is a search method in which a causal relationship is traced in a forward direction from a problem to a solution and then a causal relationship and a specific semantic relationship are traced in a backward direction. As a result, after a solution is obtained from a certain problem, it is possible to obtain another problem simultaneously solved by the solution, a situation and a phenomenon occurring by the solution, and the like.

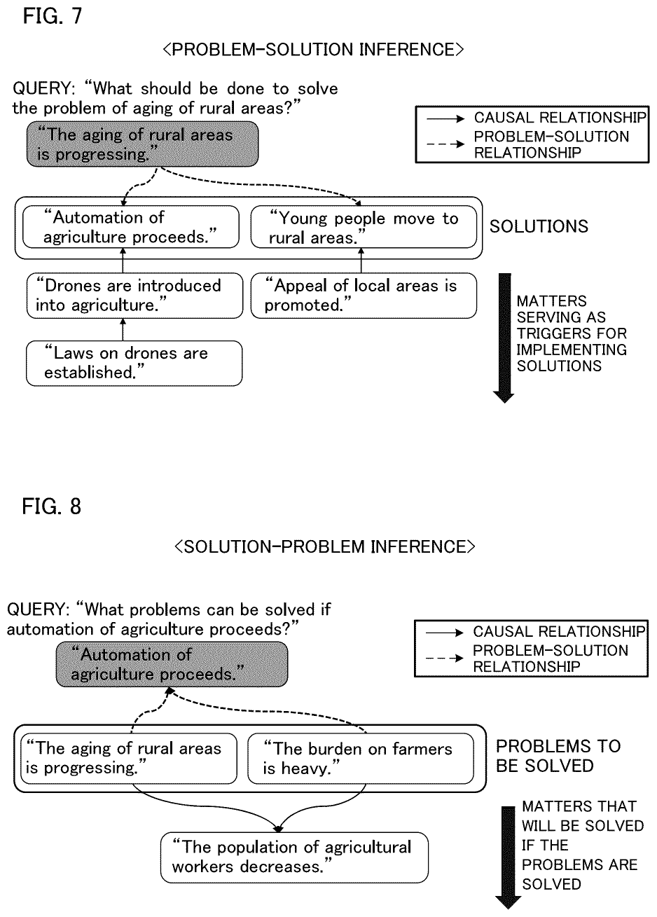

FIG. 7 illustrates an example of the problem-solution inference. Note that a solid arrow in the drawing indicates a causal relationship, and a broken arrow indicates a problem-solution relationship. In the problem-solution inference, a solution is inferred from a query, and a matter serving as a trigger for implementing the solution is further inferred. In the example of FIG. 7, a phrase “Automation of agriculture proceeds.” is obtained based on a query “The aging of rural areas is progressing.”. Furthermore, a phrase “Drones are introduced into agriculture.” is obtained based on the phrase “Automation of agriculture proceeds.”, and a phrase “Laws on drones are established.” is obtained based on the phrase “Drones are introduced into agriculture.”.

### (C2) Solution-Problem Inference

The solution-problem inference is an inference type for a query such as “What problem is solved by XX?”, and is a search method in which a causal relationship is traced in a backward direction from a solution to a problem, and then a causal relationship and a specific semantic relationship are traced in a forward direction. Thereby, for example, after a problem to be solved by a certain solution is obtained from the solution, it is possible to further obtain another solution to solve the problem.

FIG. 8 illustrates an example of the solution-problem inference. Note that a solid arrow in the drawing indicates a causal relationship, and a broken arrow indicates a problem-solution relationship. In the solution-problem inference, a problem to be solved is inferred by a query, and a matter that will be solved in a case where the problem is solved is further inferred. In the example of FIG. 8, a phrase “The aging of rural areas is progressing.” is obtained based on a query “The aging of rural areas is progressing.”. Furthermore, a phrase “The population of agricultural workers decreases.” is obtained based on the phrase “The aging of rural areas is progressing.”.

### (D) Sub-Classification Designation Inference

The subcategory designation inference is a search method of limiting a causal relationship used at the time of inference to a causal relationship of a specific subcategory. That is, the subcategory designation inference makes a search considering or excluding a causal relationship belonging to a specific subcategory among the plurality of causal relationships included in the causal relationship knowledge. For example, as described above, it is assumed that the causal relationship knowledge stored in the causal relationship DB 21 includes three subcategories: the “causal relationship in a narrow sense”, the “problem-solution relationship”, and the “chronological order relationship”. In a case where the user designates “considering only the problem-solution relationship” as an inference condition based on the query, the question answering device 100 makes a search by tracing only a problem-solution relationship and a specific semantic relationship related thereto in the causal relationship knowledge stored in the causal relationship DB 21. On the contrary, in a case where the user designates “excluding the problem-solution relationship” as an inference condition based on the query, the question answering device 100 makes a search by tracing only a causal relationship other than the problem-solution relationship and a specific semantic relationship related thereto in the causal relationship knowledge stored in the causal relationship DB 21.

Next, a method of determining an inference type by the inference type determination unit 24 will be described. The inference type determination unit 24 determines an inference type by performing text analysis on the query input by the user. Specifically, in a first method, the inference type determination unit 24 determines an inference type by using a rule-based machine learning model. For example, the inference type determination unit 24 generates a rule-based machine learning model for determining an inference type based on words, symbols, and the like included in a phrase constituting the query, and determines an inference type by using the machine learning model. In a second method, the inference type determination unit 24 generates a classification model that receives a phrase of the query as an input and outputs an inference type related to the phrase, and determines an inference type using the classification model. This classification model may use, for example, a neural network. In a third method, the inference type determination unit 24 determines an inference type based on whether a specific word (for example, “why”, “what should I do”, or the like) or a symbol (“?” or the like) is included at the beginning or end of a sentence constituting the query. The inference type determination unit 24 outputs the inference type determined by any of the methods to the inference unit 25.

The inference unit 25 infers an answer to the query by using the starting point phrase determined by the starting point phrase determination unit 23 and the inference type determined by the inference type determination unit 24. Specifically, the inference unit 25 searches for a phrase having a causal relationship with the query by tracing a causal relationship and a specific semantic relationship starting from the starting point phrase as a starting point in the causal relationship knowledge including a specific semantic relationship pair as illustrated in FIG. 4. The inference unit 25 then outputs a phrase of a node at which the causal relationship and the specific semantic relationship cannot be traced (hereinafter, such a node is also referred to as an “end node”) as an answer to the query. As described above, in a case where the starting point phrase determination unit 23 determines a plurality of starting point phrases for the query, the inference unit 25 may individually search for phrases by using each starting point phrase as a starting point, and output phrases of end nodes related to the individual searches as answers.

### Inference Processing

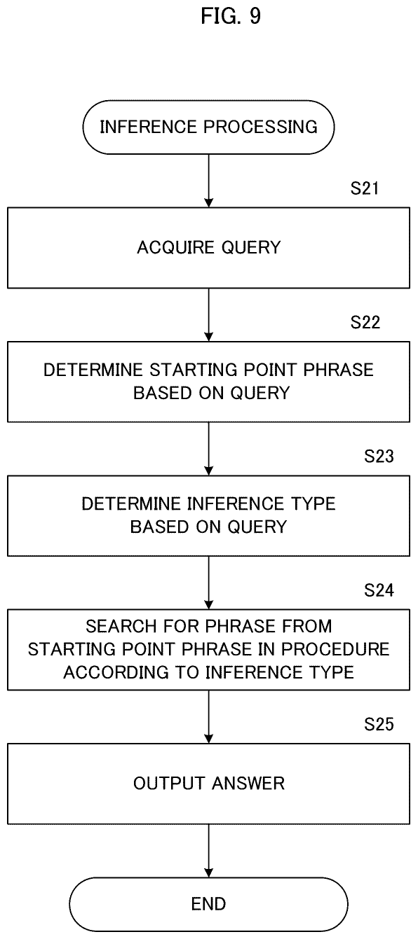

FIG. 9 is a flowchart of inference processing by the question answering device 100 according to the first example embodiment. This processing is implemented by the processor 13 illustrated in FIG. 2 executing a program prepared in advance and operating as each element illustrated in FIG. 3.

First, the question answering device 100 acquires a query input by a user (step S21). Next, the starting point phrase determination unit 23 determines a starting point phrase related to the input query in causal relationship knowledge stored in the causal relationship DB 21 (step S22). Next, the inference type determination unit 24 determines an inference type related to the input query (step S23).

Next, the inference unit 25 searches for a phrase by tracing a causal relationship and a specific semantic relationship included in the causal relationship knowledge with the starting point phrase determined in step S22 as a starting point in accordance with the inference type determined in step S23 (step S24). Specifically, the inference unit 25 traces the causal relationship based on the causal relationship knowledge. At the same time, the semantic relationship determination unit 22 determines whether there is a phrase having a specific semantic relationship with the phrase at the current position in the search. In a case where there is a phrase having a specific semantic relationship with the phrase at the current position, the inference unit 25 makes the search also by tracing the specific semantic relationship. That is, the inference unit 25 searches for a phrase in consideration of the specific semantic relationship extracted by the semantic relationship determination unit 22. In a case where there is no causal relationship or specific semantic relationship to be traced, that is, in a case where the current position of the search reaches an end node, the inference unit 25 outputs a phrase of the end node as an answer to the query (step S25). The inference processing is then terminated.

As described above, in the first example embodiment, an answer to a query is inferred for phrases having specific semantic relationships such as an entailment, a synonym, and a similar meaning in addition to causal relationship knowledge prepared in advance, and thus it is possible to infer the answer based on more causal relationship knowledge. In addition, a starting point phrase and an inference type are determined based on the query input by the user, and a phrase is searched for from the determined starting point phrase in accordance with the determined inference type, which makes it possible to infer the answer by a method suitable for the input query.

### Second Example Embodiment

Next, a second example embodiment of the present disclosure will be described. In the first example embodiment, the question answering device 100 performs inference for a query while extracting a specific semantic relationship pair by using the semantic relationship determination unit 22. Instead, in the second example embodiment, a question answering device extracts a specific semantic relationship in advance with respect to causal relationship knowledge stored in a causal relationship DB, and creates a relationship graph including a causal relationship and the specific semantic relationship. In the second example embodiment, the question answering device outputs an answer graph to be described later as an answer to a query. Except for these points, the second example embodiment is basically similar to the first example embodiment. Specifically, an overall configuration and a hardware configuration of a question answering device 100x according to the second example embodiment are similar to the overall configuration of the first example embodiment illustrated in FIG. 1 and the hardware configuration illustrated in FIG. 2, and thus the description thereof is omitted.

### Functional Configuration

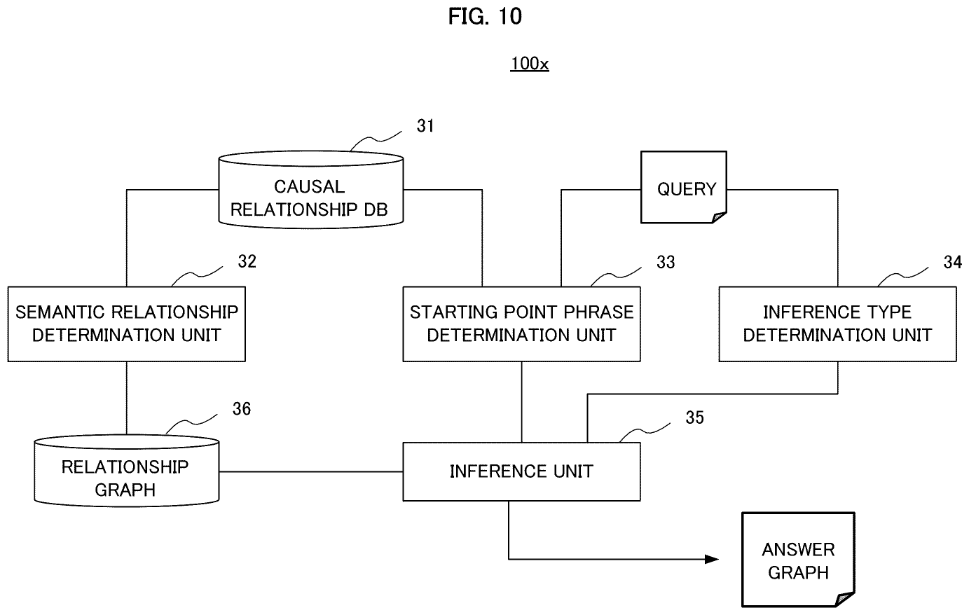

FIG. 10 is a block diagram illustrating a functional configuration of the question answering device 100x according to the second example embodiment. As illustrated, the question answering device 100x according to the second example embodiment includes a causal relationship DB 31, a semantic relationship determination unit 32, a starting point phrase determination unit 33, an inference type determination unit 34, an inference unit 35, and a relationship graph 36. The causal relationship DB 31, the starting point phrase determination unit 33, and the inference type determination unit 34 are basically similar to the causal relationship DB 21, the starting point phrase determination unit 23, and the inference type determination unit 24 in the first example embodiment, and thus description thereof is omitted.

The semantic relationship determination unit 32 extracts specific semantic relationship pairs for the entire causal relationship knowledge stored in the causal relationship DB 31 as preprocessing. The semantic relationship determination unit 32 then generates the relationship graph 36 in which the extracted specific semantic relationship pairs are added to the causal relationship knowledge. As illustrated in FIG. 4, the generated relationship graph 36 has a graph structure including causal relationships and specific semantic relationships. That is, the relationship graph 36 is a directed graph in which each phrase is indicated by a node and a causal relationship or a specific semantic relationship is indicated by an edge. As described above, in the second example embodiment, the relationship graph 36 in which the specific semantic relationship pairs are added to the causal relationship knowledge is generated as preprocessing, and in a case where a user inputs a query, an answer is inferred with reference to the relationship graph 36. Therefore, it is possible to reduce the operation load of inference in response to the query input and shorten the time required to output the answer.

When the user inputs a query, the starting point phrase determination unit 33 determines a starting point phrase, and the inference type determination unit 34 determines an inference type, similarly to the first example embodiment. The inference unit 35 then searches for a phrase by tracing a causal relationship and a specific semantic relationship with the starting point phrase as a starting point in the relationship graph 36. Note that in the first example embodiment, the inference unit 25 outputs a phrase of an end node at which the causal relationship and the specific semantic relationship cannot be traced as an answer to the query. Meanwhile, in the second example embodiment, the inference unit 35 outputs a graph indicating a portion in which the causal relationship and the specific semantic relationship are traced in the search for a phrase (such a graph is referred to as an “answer graph”) as an answer to the query.

### Inference Processing

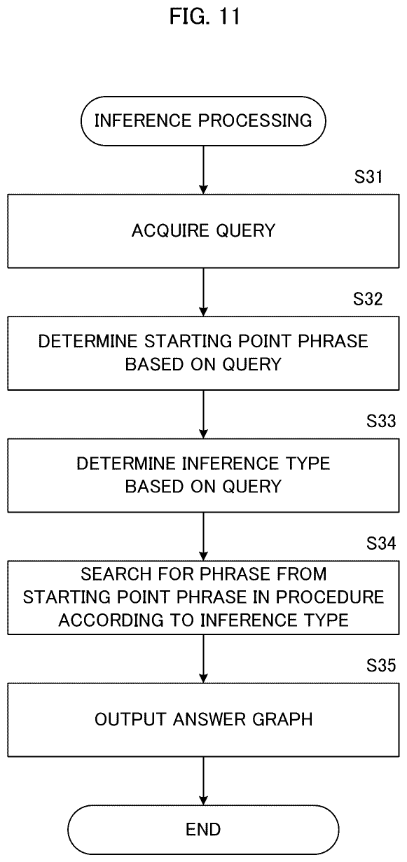

FIG. 11 is a flowchart of inference processing by the question answering device 100x according to the second example embodiment. This processing is implemented by the processor 13 illustrated in FIG. 2 executing a program prepared in advance and operating as each element illustrated in FIG. 10. In the second example embodiment, it is assumed that, as preprocessing, the semantic relationship determination unit 32 has extracted specific semantic relationship pairs between phrases included in the causal relationship DB 31, and has generated the relationship graph 36 including causal relationships and specific semantic relationships.

First, the question answering device 100x acquires a query input by a user (step S31). Next, the starting point phrase determination unit 33 determines a starting point phrase related to the input query in causal relationship knowledge stored in the causal relationship DB 31 (step S32). Next, the inference type determination unit 34 determines an inference type related to the input query (step S33).

Next, the inference unit 35 searches for a phrase by tracing a causal relationship and a specific semantic relationship in the relationship graph 36 with the starting point phrase determined in step S32 as a starting point in accordance with the inference type determined in step S33 (step S34). Next, in a case where there is no causal relationship or specific semantic relationship to be traced, the inference unit 35 outputs a portion traced in the search from the relationship graph 36 as an answer graph (step S35). As a result, the answer graph is presented to the user. The inference processing is then terminated.

### Display Example

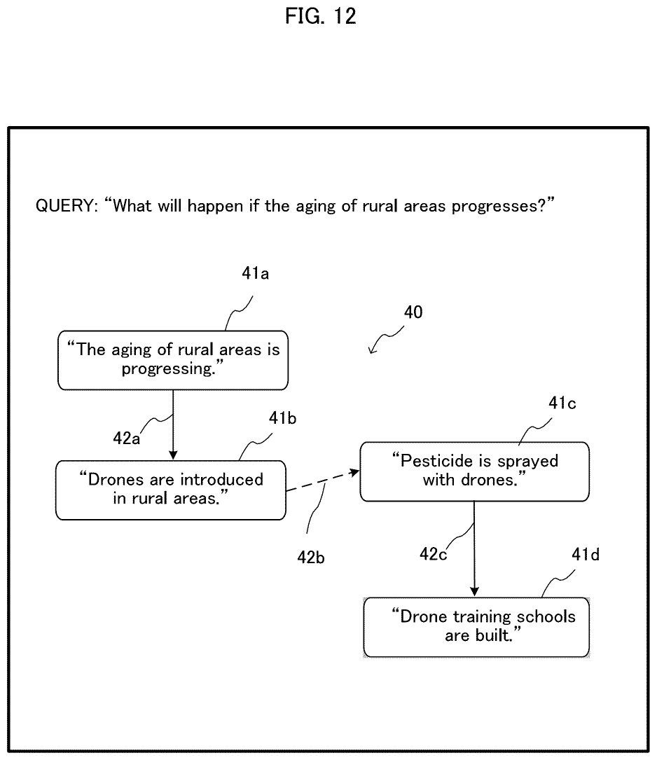

FIG. 12 illustrates a display example of the answer graph output by the inference unit 35. In this example, a query “What will happen if the aging of rural areas progresses?” is input by the user, and an answer graph 40 for this query is displayed. The answer graph 40 is obtained by cutting out a portion traced by the inference unit 35 in the search for a phrase in the relationship graph 36 used for the inference by the inference unit 35. Specifically, in the example of FIG. 12, for the query “What will happen if the aging of rural areas progresses?”, the starting point phrase determination unit 33 determines a node 41a as a starting point phrase, and the inference unit 35 proceeds from the node 41a to a node 41b through an edge 42a indicating a causal relationship, proceeds to a node 41c through an edge 42b indicating a specific semantic relationship, further proceeds to an end node 41d through an edge 42c indicating a causal relationship, and terminates the search. As described above, in the second example embodiment, the answer graph indicating the portion traced by the inference unit 35 in the relationship graph 36 at the time of inference is displayed as an answer to the query, and thus the user can know how the answer has been generated.

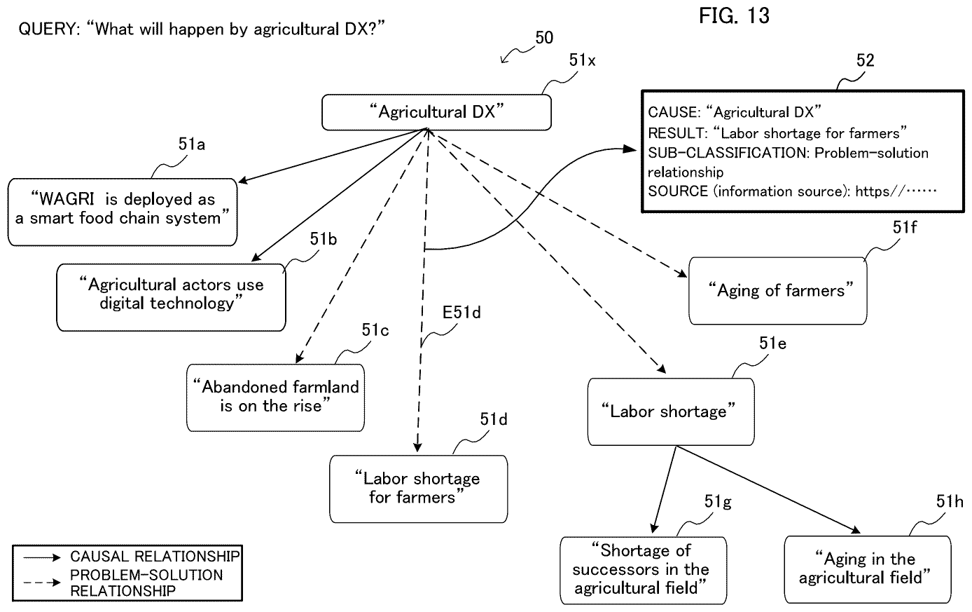

FIG. 13 illustrates another display example of the answer graph output by the inference unit 25. In this example, a query “What will happen by agricultural DX?” is input by the user, and an answer graph 50 for this query is displayed. Specifically, in the example of FIG. 13, first, the inference unit 35 determines a starting point phrase 51x based on the query “What will happen by agricultural DX?”. Next, the inference unit 35 searches for nodes 51a and 51b each having a causal relationship with the starting point phrase 51x and displays these phrases. The inference unit 35 also searches for nodes 51c to 51f each having a problem-solution relationship with the starting point phrase 51x, further searches for nodes 51g and 51h each having a causal relationship with the node 51e, and displays these phrases.

As described above, in a case where source information of each causal relationship is included as additional information in the causal relationship knowledge stored in the causal relationship DB 31, the question answering device 100x may display the associated source information in response to the designation of the user. For example, in a case where the user clicks an edge indicating a causal relationship in the displayed answer graph, the question answering device 100x may display source information of the causal relationship knowledge related to the edge (for example, URL of a Web document describing the information) in a pop-up or the like. In the display example of FIG. 13, as a result of the user clicking an edge E51d connecting the nodes 51x and 51d, the source (information source) of the causal relationship knowledge indicated by the edge E51d is displayed in an answer 52.

Moreover, in a case where a subcategory of each causal relationship is included as additional information in the causal relationship knowledge stored in the causal relationship DB 31, the question answering device 100x may display the subcategory in response to the designation of the user. For example, in a case where the user clicks an edge indicating a causal relationship in the displayed answer graph, the question answering device 100x may display the subcategory of the causal relationship related to the edge (for example, “chronological order relationship” or the like) in a pop-up or the like. In the display example of FIG. 13, as a result of the user clicking the edge E51d connecting the nodes 51x and 51d, the “problem-solution relationship”, which is the subcategory of the causal relationship of the edge E51d, is displayed in the answer 52.

The user may give an instruction to search for a phrase related to the query by selecting a node included in the answer graph. In this case, the question answering device 100x makes a search by using the node selected by the user as the starting point phrase.

Moreover, the user may be allowed to input an evaluation for the answer or the question answering device 100x to the displayed answer graph. For example, in a case where an inference type is determined by the inference type determination unit 34 based on a rule, the rule used for determining an inference type may be corrected based on the evaluation input of the user. In a case where an inference type is determined by a machine learning model, the evaluation input of the user may be adopted as learning data, and the model for determining an inference type may be relearned.

The user may be allowed to input correction of the causal relationship knowledge to the displayed answer graph. For example, on the displayed answer graph, the user may be allowed to add a new edge or modify the direction of an edge (arrow). As a result, it is possible to correct the causal relationship knowledge used for inference based on the input of the user. Also, in a case where a causal relationship graph indicating causal relationship knowledge is generated by machine learning, the causal relationship graph may be relearned by using the input of the user

As described above, the second example embodiment prepares in advance the relationship graph 36 to which phrases having specific semantic relationships such as an entailment, a synonym, and a similar meaning are added in addition to the causal relationship knowledge prepared in advance, and thus a processing load from the input of a query by the user to the output of an answer is reduced, and inference can be performed in a short time. In addition, an answer graph indicating a causal relationship and a specific semantic relationship used in inference is displayed as an answer to the query, and thus the user can know the background of the answer or the grounds on which the answer has been obtained.

### Third Example Embodiment

Next, a third example embodiment of the present disclosure will be described. In the third example embodiment, the question answering devices according to the first and second example embodiments are applied to medical question answering.

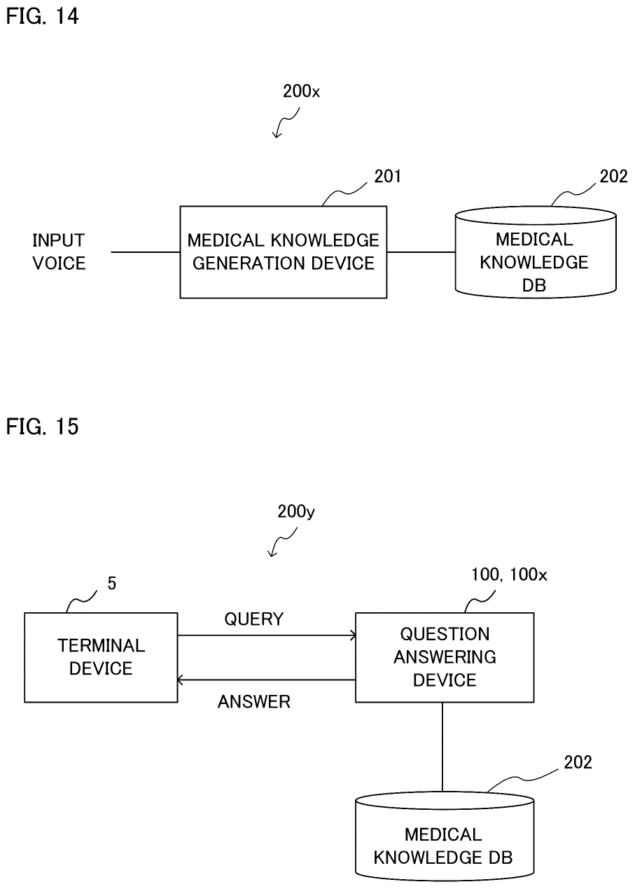

FIG. 14 illustrates a configuration of a medical knowledge generation system according to the third example embodiment. As illustrated, a medical knowledge generation system 200x includes a medical knowledge generation device 201 and a medical knowledge DB 202. The medical knowledge generation device 201 receives an input of a voice of a doctor or a nurse at the time of diagnosis of a patient. The medical knowledge generation device 201 includes medical knowledge generation AI. The medical knowledge generation AI has a function of converting an input voice into characters and summarizing the contents, a function of automatically generating a medical record based on the characters of the summary, and a function of constructing causal relationship knowledge from the medical record. The medical knowledge generation device 201 generates causal relationship knowledge regarding medical care from the voice input by the doctor or the nurse by using the medical knowledge generation AI, and stores the causal relationship knowledge in the medical knowledge DB 202 as medical knowledge.

Specifically, using the medical knowledge generation AI, the medical knowledge generation device 201 generates the medical knowledge as follows. In a case where a doctor makes a diagnosis on a patient, the medical knowledge generation AI records a voice of the doctor or a nurse. The medical knowledge generation AI converts the voice of the doctor or the nurse into characters. The characters reflect the contents of the diagnosis made by the doctor on the patient. Next, the medical knowledge generation AI summarizes the characters obtained by the character conversion. This summary is a brief summary of the contents of the diagnosis made by the doctor on the patient. Next, the medical knowledge generation AI automatically generates a medical record of the patient based on the characters obtained by the summary. This medical record describes basic information of the patient, the contents of the diagnosis, a treatment method, and the like.

Next, the medical knowledge generation AI constructs causal relationship knowledge from the generated medical record by using a predetermined machine learning algorithm. At this time, the causal relationship knowledge may include not only the past medical record of the user but also published medical information. In this way, the causal relationship knowledge regarding medical care is generated and accumulated in the medical knowledge DB 202 as medical knowledge.

FIG. 15 illustrates a configuration of a question answering system according to the third example embodiment. A question answering system 200y makes an AI diagnosis using a question answering device to support a diagnosis of a patient made by a doctor. The question answering system 200y is obtained by applying the question answering device 100 according to the first example embodiment or the question answering device 100x according to the second example embodiment.

In the diagnosis of the patient, the doctor inputs a query to the question answering device 100 or 100x using the terminal device 5. The question answering device 100 or 100x generates an answer to the query of the doctor by using the medical knowledge DB 202 in which causal relationship knowledge regarding medical care is accumulated, and transmits the answer to the terminal device 5 of the doctor. Using the technique of the first or second example embodiment, the question answering device 100 or 100x transmits, as an answer, a cause or diagnosis result of the symptom of the patient, which has been inferred and optimized by use of a past medical record of the user, published medical information, or the like, to the terminal device 5 of the doctor. The doctor can make a diagnosis of the patient with reference to the answer of the question answering device 100 or 100x.

### Fourth Example Embodiment

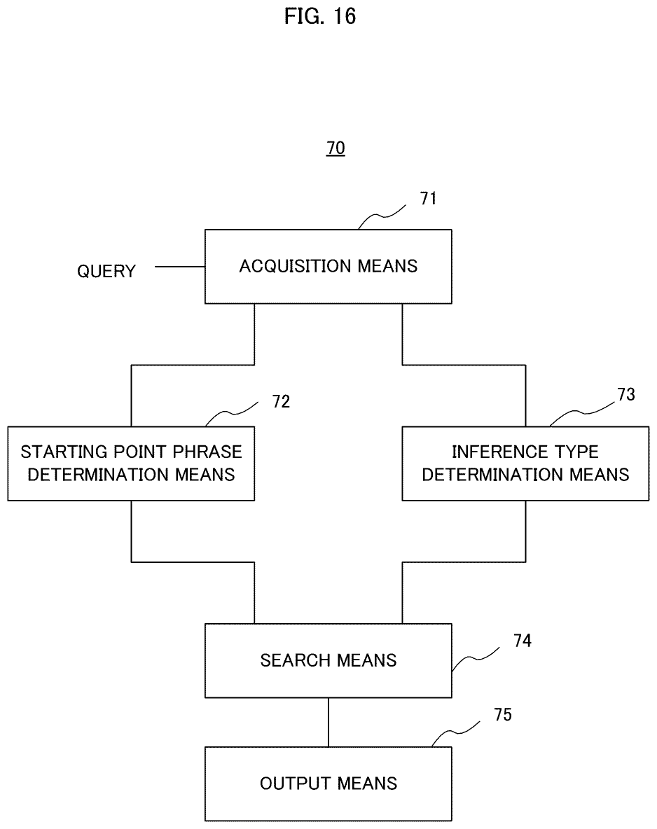

FIG. 16 is a block diagram illustrating a functional configuration of a question answering device according to a fourth example embodiment. A question answering device 70 includes an acquisition means 71, a starting point phrase determination means 72, an inference type determination means 73, a search means 74, and an output means 75.

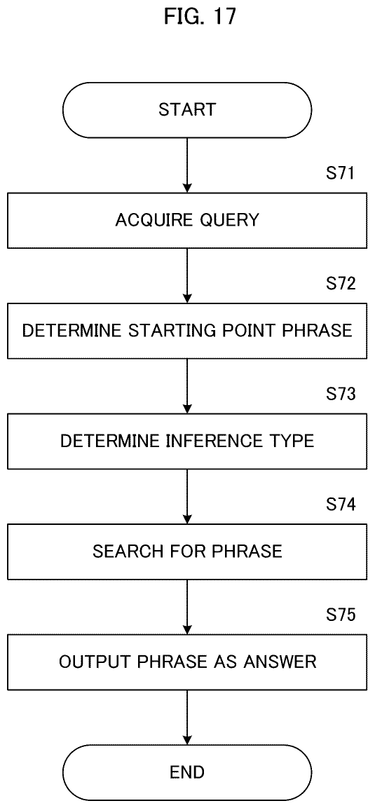

FIG. 17 is a flowchart of processing by the question answering device 70 according to the fourth example embodiment. The acquisition means 71 acquires a query (step S71). The starting point phrase determination means 72 determines a starting point phrase related to the query from a plurality of phrases included in causal relationship data (step S72). The inference type determination means 73 determines an inference type based on the query (step S73). The search means 74 searches for a phrase by tracing a causal relationship included in the causal relationship data from the starting point phrase in a search procedure according to the determined inference type (step S74). The output means 75 outputs the phrase obtained by the search as an answer to the query (step S75).

According to the question answering device 70 according to the fourth example embodiment, it is possible to efficiently generate and output an answer to a question based on causal relationship knowledge.

### MODIFICATIONS

### First Modification

In the above example embodiments, the specific semantic relationship pairs are added to the causal relationship knowledge, and in a case where an answer to a query is inferred, a phrase is searched for by tracing a causal relationship and a specific semantic relationship. Instead, the question answering device may search for a phrase by tracing only a causal relationship without considering a specific semantic relationship.

### Second Modification

In the above example embodiments, as illustrated in FIG. 1, the terminal device 5 of the user and the question answering device 100 are connected by communication, but instead, the question answering device 100 may be configured as an independent device. In this case, the question answering device 100 may be provided with an input unit and a display unit, and the user may input a query to the input unit and display an answer to the query on the display unit.

A part or all of the example embodiments described above may also be described as the following supplementary notes, but not limited thereto.

### Supplementary Note 1

A question answering device comprising:
a query acquisition means configured to acquire a query;
a starting point phrase determination means configured to determine a starting point phrase related to the query from a plurality of phrases included in causal relationship data;
an inference type determination means configured to determine an inference type based on the query;
a search means configured to search for a phrase by tracing a causal relationship included in the causal relationship data from the starting point phrase in a search procedure according to the determined inference type; and
an output means configured to output a phrase obtained by the search as an answer to the query.

### Supplementary Note 2

The question answering device according to supplementary note 1, comprising
a semantic relationship determination means configured to extract a specific semantic relationship between a plurality of phrases included in the causal relationship data, wherein
the search means makes the search by tracing a causal relationship and a specific semantic relationship included in the causal relationship data.

### Supplementary Note 3

The question answering device according to supplementary note 2, wherein the specific semantic relationship includes at least one of an entailment relationship, a synonym relationship, and a similar meaning relationship between phrases.

### Supplementary Note 4

The question answering device according to supplementary note 1, wherein the starting point phrase determination means determines, as the starting point phrase, a phrase having a meaning closer to a meaning of the phrase of the query than a predetermined standard among the plurality of phrases included in the causal relationship data.

### Supplementary Note 5

The question answering device according to supplementary note 1, wherein the inference type determination means analyzes the phrase of the query and determines an inference type related to an inference requested by the phrase.

### Supplementary Note 6

The question answering device according to supplementary note 1, wherein the inference type includes a first inference type indicating a search procedure of tracing the causal relationship data in a forward direction of a causal relationship, a second inference type indicating a search procedure of tracing the causal relationship data in a backward direction of a causal relationship, and a third inference type indicating a search procedure combining a search of tracing the causal relationship data in the forward direction of a causal relationship and a search of tracing the causal relationship data in the backward direction.

### Supplementary Note 7

The question answering device according to supplementary note 6, wherein
the causal relationship data includes data indicating a subcategory of a causal relationship, and
the inference type includes a fourth inference type of limiting a search target to a causal relationship related to a predetermined subcategory in the causal relationship data.

### Supplementary Note 8

The question answering device according to supplementary note 1, wherein the output means outputs, as the answer to the query, a graph indicating a portion targeted in the search in the causal relationship data.

### Supplementary Note 9

The question answering device according to supplementary note 1, wherein
the query is a question related to a diagnosis of a patient,
the causal relationship data includes causal relationship knowledge regarding medical care constructed from a past medical record of a user and published medical information, and
the output means outputs an optimized treatment policy as the answer.

### Supplementary Note 10

A question answering method executed by a computer, the question answering method comprising:
acquiring a query;
determining a starting point phrase related to the query from a plurality of phrases included in causal relationship data;
determining an inference type based on the query;
searching for a phrase by tracing a causal relationship included in the causal relationship data from the starting point phrase in a search procedure according to the determined inference type; and
outputting a phrase obtained by the search as an answer to the query.

### Supplementary Note 11

A recording medium recording a program for causing a computer to execute processing comprising:
acquiring a query;
determining a starting point phrase related to the query from a plurality of phrases included in causal relationship data;
determining an inference type based on the query;
searching for a phrase by tracing a causal relationship included in the causal relationship data from the starting point phrase in a search procedure according to the determined inference type; and
outputting a phrase obtained by the search as an answer to the query.

While the disclosure has been described with reference to the example embodiments and examples, the disclosure is not limited to the above example embodiments and examples. It will be understood by those of ordinary skill in the art that various changes in form and details may be made therein without departing from the spirit and scope of the present disclosure as defined by the claims.

### DESCRIPTION OF SYMBOLS

5 Terminal device
13 Processor
21, 31 Causal relationship DB
22, 32 Semantic relationship determination unit
23, 33 Starting point phrase determination unit
24, 34 Inference type determination unit
25, 35 Inference unit
36 Relationship graph

What is claimed is:

1. A question answering device comprising:
at least one memory configured to store instructions; and
at least one processor configured to execute the instructions to:
acquire a query;
determine a starting point phrase related to the query from a plurality of phrases included in causal relationship data;
determine an inference type based on the query;
search for a phrase by tracing a causal relationship included in the causal relationship data from the starting point phrase in a search procedure according to the determined inference type; and
output a phrase obtained by the search as an answer to the query.

2. The question answering device according to claim 1, comprising
extract a specific semantic relationship between a plurality of phrases included in the causal relationship data, wherein
the processor makes the search by tracing a causal relationship and a specific semantic relationship included in the causal relationship data.

3. The question answering device according to claim 2, wherein the specific semantic relationship includes at least one of an entailment relationship, a synonym relationship, and a similar meaning relationship between phrases.

4. The question answering device according to claim 1, wherein the processor determines, as the starting point phrase, a phrase having a meaning closer to a meaning of the phrase of the query than a predetermined standard among the plurality of phrases included in the causal relationship data.

5. The question answering device according to claim 1, wherein the processor analyzes the phrase of the query and determines an inference type related to an inference requested by the phrase.

6. The question answering device according to claim 1, wherein the inference type includes a first inference type indicating a search procedure of tracing the causal relationship data in a forward direction of a causal relationship, a second inference type indicating a search procedure of tracing the causal relationship data in a backward direction of a causal relationship, and a third inference type indicating a search procedure combining a search of tracing the causal relationship data in the forward direction of a causal relationship and a search of tracing the causal relationship data in the backward direction.

7. The question answering device according to claim 6, wherein
the causal relationship data includes data indicating a subcategory of a causal relationship, and
the inference type includes a fourth inference type of limiting a search target to a causal relationship related to a predetermined subcategory in the causal relationship data.

8. The question answering device according to claim 1, wherein the processor outputs, as the answer to the query, a graph indicating a portion targeted in the search in the causal relationship data.

9. The question answering device according to claim 1, wherein
the query is a question related to a diagnosis of a patient,
the causal relationship data includes causal relationship knowledge regarding medical care constructed from a past medical record of a user and published medical information, and
the processor outputs an optimized treatment policy as the answer.

10. A question answering method executed by a computer, the question answering method comprising:
acquiring a query;
determining a starting point phrase related to the query from a plurality of phrases included in causal relationship data;
determining an inference type based on the query;
searching for a phrase by tracing a causal relationship included in the causal relationship data from the starting point phrase in a search procedure according to the determined inference type; and
outputting a phrase obtained by the search as an answer to the query.

11. A non-transitory computer readable recording medium recording a program for causing a computer to execute processing comprising:
acquiring a query;
determining a starting point phrase related to the query from a plurality of phrases included in causal relationship data;
determining an inference type based on the query;
searching for a phrase by tracing a causal relationship included in the causal relationship data from the starting point phrase in a search procedure according to the determined inference type; and
outputting a phrase obtained by the search as an answer to the query.

12. The question answering device according to claim 1, wherein the output unit selects an optimal answer to the query from the phrase obtained by the search based on machine learning for decision making.
